SEQUENCE LISTING (1 sequence)

SEQ ID NO: 1 - protein, 179 aa, homo sapiens
DIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQL

(19) United States
(12) Patent Application Publication
(10) Pub. No.: US 2020/0255531 A1
(43) Pub. Date: Aug. 13, 2020

(54) METHOD FOR MODULATION OF TUMOR ASSOCIATED MYELOID CELLS AND ENHANCING IMMUNE CHECKPOINT BLOCKADE

(71) Applicants: MACKAY MEMORIAL HOSPITAL, Taipei (TW); ASCENDO BIOTECHNOLOGY, INC., Grand Cayman (KY)

(72) Inventors: Yen-Ta Lu, Taipei (TW); Chia-Ming Chang, Taipei (TW); I-Fang Tsai, Taipei (TW); Meng-Ping Lu, Taipei (TW); Haishan Jang, Taipei (TW); Ping-Yen Huang, Taipei (US)

(73) Assignees: MACKAY MEMORIAL HOSPITAL, Taipei (TW); ASCENDO BIOTECHNOLOGY, INC., Grand Cayman (KY)

(21) Appl. No.: 16/761,259

(22) PCT Filed: Nov. 5, 2018
(86) PCT No.: PCT/US2018/059247

§ 371 Date: May 3, 2020

Related U.S. Application Data
(60) Provisional application No. 62/581,632, filed on Nov. 3, 2017.

(51) Int. Cl. C07K 16/28; A61K 31/337

(52) U.S. Cl. CPC C07K 16/2845; A61K 31/337; C07K 16/2818

(57) ABSTRACT

The present invention relates to methods for modulating immune response based on binding I-domain of CD11b on the tumor associated myeloid cells (TAMCs) in the tumor microenvironment. Particularly, binding to I-domain of CD11b with anti-CD11b-I-domain antibody triggers immunostimulatory environment that have one or more of the following effects in the tumor microenvironment: increase the inflammatory cytokine in the tumor microenvironment, decrease the population of IDO+ myeloid suppresser cells, up-regulate M1 marker over M2 marker on the tumor associated macrophage, increase M1:M2 tumor associated macrophage ratio, promote differentiation of dendritic cells (DC), nature killer dendritic cells (NKDC), and plasmacytoid dendritic cells (pDC), increase population of 4−1BB+PD−1+ neoantigen specific CD8 T cells. Converting cold (non-inflamed) to hot (inflamed) tumor by binding to I-domain of CD11b with anti-CD11b-I-domain antibody allows enhanced effectiveness of immune response modulator.

### FIELD OF THE INVENTION

The present invention relates to methods for modulating immune responses, particularly to methods involving binding to the I-domain of CD11b.

### BACKGROUND OF THE INVENTION

Integrin alpha M (CD11b, CR3A, or ITGAM) is one protein subunit that forms the heterodimeric integrin alpha-M beta-2 (αMβ2) molecule that expresses on the surface of numerous innate immune cells, including monocytes, granulocytes, macrophage, dendritic cells, NK cells, nature killer dendritic cells, plasmacytoid dendritic cells, and myeloid-derived suppressor cells (MDSCs).

CD11b consists of a large extracellular region, a single hydrophobic transmembrane domain, and a short cytoplasmic tail. The extracellular region of the CD11b comprises a β-propeller domain, a thigh domain, a calf-1 domain, and a calf-2 domain. The I-domain of CD11b consists of around 179 amino acids inserted in the β-propeller domain. The I-domain is the binding site for various ligands (e.g., iC3b, fibrinogen, ICAM-I, and CD40L, etc.) and mediates inflammation, by regulating cell adhesion, migration, chemotaxis, and phagocytosis.

It has been shown that ligation of CD11b could facilitate the development of peripheral tolerance by inhibiting T helper 17 (Th17) differentiation. In addition, active CD11b expressed on antigen-presenting cells (dendritic cells and macrophages) can directly inhibit full T cell activation. Results from recent research show that CD11b plays a critical role in inflammation by modulating Toll-Like Receptor (TLR) responses. High avidity ligation of CD11b-I-domain leads to rapid inhibition of TLR signaling by promoting degradation of myeloid differentiation primary response protein 88 (MyD88) and TTR-domain-containing adapter-inducing interferon-β (TRIF). Therefore, integrin αMβ2 may serve as a negative regulator of innate immune responses.

Immune checkpoint blockade drugs, such as anti-PD1, anti-PDL1, and anti-CTLA4 antibodies, provide tumor destructive immune responses and can elicit durable clinical responses in cancer patients. However, these drugs work best in “hot” tumors (i.e., those that are inflamed, with high mutagenic burden, and capable of attracting neoantigen specific T-cell infiltration). In contrast, “cold” tumors (i.e., those that are non-inflamed, with low mutagenic burden, and incapable of attracting neoantigen specific T-cell infiltration) are typically less responsive to immune checkpoint blockade therapy.

Tumor microenvironment is a complex environment, upon which tumors depend for sustained growth, invasion, and metastasis. Many studies have shown that tumor-associated myeloid cells (TAMCs) are major components of the immune cells in the tumor microenvironment, and TAMCs are believed to promote, directly or indirectly, tumor progression. TAMCs in the tumor microenvironment are composed of myeloid-derived suppresser cells (MDSCs), tumor-associated macrophages (TAMs), neutrophils, mast cells, and dendritic cells. These cells contribute to the suppression of T cell functions, and such suppression correlates with immune checkpoint blocking resistance. Therefore, these TAMCs may be targets of new cancer immunotherapy.

### SUMMARY OF THE INVENTION

One aspect of the invention relates to methods for modulating immune responses. A method in accordance with one embodiment of the invention comprises modulating an immune response, comprising administering a pharmaceutical composition to a subject in need thereof, wherein the pharmaceutical composition comprises a reagent that binds specifically to the I-domain of CD11b on cells, such as tumor-associated myeloid cells (TAMCs). The reagent may be an antibody that binds the I-domain of CD11b. The I domain of CD11b has major recognition sites for various adhesion ligands (M. S. Diamond et al., J. Cell Biol., 120 (4): 1031). The fact that binding to the I-domain of CD11b, which is known for adhesion functions, can modulate immune responses is truly unexpected.

In accordance with some embodiments of the invention, the pharmaceutical composition for modulating immune responses may further comprise another immune response modulator, such as an immune checkpoint blockade drug. The immune checkpoint blockade drug is a reagent that binds specifically to CTLA4, such as an anti-CTLA4 antibody.

In accordance with some embodiments of the invention, the pharmaceutical composition further comprises an immune checkpoint blockade drug. The immune checkpoint blockade drug is a reagent that binds specifically to PD1, such as an anti-PD1 antibody.

In accordance with some embodiments of the invention, the pharmaceutical composition further comprises an immune checkpoint blockade drug. The immune checkpoint blockade drug is a reagent that binds specifically to PDL1, such as an anti-PDL1 antibody.

In accordance with some embodiments of the invention, the pharmaceutical composition further comprises an immune checkpoint blockade drug. The immune checkpoint blockade drug is a reagent that binds specifically to OX40 (i.e., CD134), such as an anti-OX40 antibody.

In accordance with some embodiments of the invention, the pharmaceutical composition further comprises an immune checkpoint blockade drug. The immune checkpoint blockade drug is a reagent that binds specifically to CD40, such as an anti-CD40 antibody.

Embodiments of the invention involve specific binding of a reagent to the I-domain of CD11b to modulate the immune responses. As a result, tumor microenvironment is changed from that of a cold tumor to that of a hot tumor, rendering the tumor more susceptible to various therapeutic treatments, including chemotherapy and radiation therapy. Thus, some embodiments of the invention involve combination therapies using a reagent that binds specifically to the I-domain of CD11b and another cancer therapeutic modality (e.g., chemotherapeutic agent or radio therapy). Examples of chemotherapeutic agents may include taxol or other chemotherapeutics.

Other aspect of the invention will become apparent with the following description and the associated drawings.

### BRIEF DESCRIPTION OF THE DRAWINGS

FIG. 1 shows cytokine profiles in B16F10 tumor tissue fluids after anti-CD11b-I-domain antibody treatment. C57/BL6 mice were injected subcutaneously with 2×10⁵ B16F10 cells. When tumor volumes were approximately 500 mm³, mice were injected ip with either a control IgG (5 mg/kg) or an anti-CD11b-I-domain antibody (5 mg/kg). One day later, mice were sacrificed and cytokine concentrations in the tumor tissue fluids were measured using BD cytometric bead array (CBA).

FIG. 2 shows the percentage of IDO+ MDSCs following anti-CD11b-I-domain antibody treatment. Indoleamine 2,3-dioxygenase (IDO) expression in MDSCs stimulated with phorbol 12-myristate-13-acetate (PMA) for 24 hrs. to 72 hrs., in the presence of a control IgG or an anti-CD11b-I-domain antibody, were evaluated by cellular surface staining with anti-mouse Gr-1 FITC antibody and intracellular staining with anti-mouse IDO APC antibody. The results show that there is a time-dependent reduction of IDO+ MDSCs following the anti-CD11b-I-domain antibody treatment, as compared with treatments with the control IgG.

FIG. 3 shows the in vitro proliferation index of CD8 cells, in the presence of MDSCs and a control IgG or an anti-CD11b-I-domain antibody. MDSCs can interact with and suppress immune cells, including T cells. Here, the suppressive activity of MDSCs is assessed by their abilities to inhibit T cell activations by anti-CD3 and anti-CD28 antibodies, as observed with CD8 cell proliferation. As shown in FIG. 3, in the presence of an anti-CD11b-I-domain antibody, the T-cell suppressive abilities of MDSCs is inhibited, and CD8 cell proliferation is increased, as compared with the treatment with the control IgG.

FIG. 4 shows the effects of treatments with anti-CD11b-I-domain antibodies (e.g., 44aacb and M1/70 antibodies) on tumor associated macrophage phenotype (M1 or M2) polarization. The results show that anti-CD11b-I-domain antibody treatment significantly increase the M1 macrophage, relative to M2 macrophage. In addition, treatments with anti-CD11b-I-domain antibodies also increase dendritic cell populations, as evidenced by the increase in CD11 c and DC-SIGN dendritic cell markers.

FIG. 5 shows results of flow cytometric analyses and quantifications of M1/M2 tumor associated macrophages in the CT26 tumors after anti-CD11b-I-domain antibody treatment. Balb/c mice were injected subcutaneously with 3×10⁵ CT26 cells on day 0. When tumor volumes were approximately 50-100 mm³, mice were injected ip with either control IgG (5 mg/kg), anti-CD11b-I-domain antibody (5 mg/kg), or anti-PD-L1 antibody (5 mg/kg). Injections were repeated every three to four days. After the fourth treatment, mice were sacrificed, and tumor associated macrophages were isolated. M1 (MHC II+, CD206−) and M2 (MHC II−, CD206+) phenotypes of tumor associated macrophages were analysis by flow cytometry.

FIG. 6 shows the flow cytometric analysis and quantification of MHC II on tumor associated macrophages (TAM) in the CT26 tumors after anti-CD11b-I-domain antibody treatment. Balb/c mice were injected subcutaneously with 3×10⁵ CT26 cells on day 0. When tumor volumes were approximately 50-100 mm³, mice were injected ip with control IgG (5 mg/kg), anti-CD11b-I-domain antibody (5 mg/kg), or anti-PD-L1 antibody (5 mg/kg). Injections were repeated every three to four days. After the fourth treatment, mice were sacrificed, and tumor associated macrophages were isolated. Intensity of MHC II on tumor associated macrophages were analysis by flow cytometry. *P<0.05; **P<0.01.

FIG. 7 shows the effects of anti-CD11b-I-domain antibody and CpG combination therapy on the growth of CT26 tumor. Balb/c mice were injected subcutaneously with 3×10⁵ CT26 cells on day 0. When tumor volumes were approximately 50-100 mm³, mice (5 per group) were injected ip with either control IgG (5 mg/kg), anti-CD11b-I-domain antibody (5 mg/kg), CpG oligonucleotide (class B, ODN 1668) (50 μg), or anti-CD11b-I-domain antibody (5 mg/kg)+CpG oligonucleotide (class B, ODN 1668) (50 μg). The Second injections were repeated three days after first treatment. Tumor volumes were measured, and the results are presented as the mean±SEM.

FIG. 8 shows the effect of anti-CD11b-I-domain antibody and anti-CTLA4 antibody combination therapy on the growth of CT26 tumor. Balb/c mice were injected subcutaneously with 3×10⁵ CT26 cells on day 0. When tumor volumes were approximately 50-100 mm³, mice (5 per group) were injected ip with either control IgG (5 mg/kg), anti-CD11b -I-domain antibody (5 mg/kg), anti-CTLA4 antibody (5 mg/kg), or anti-CD11b-I-domain antibody (5 mg/kg)+anti-CTLA4 antibody (5 mg/kg). Injections were repeated every three to four days. Tumor volumes were measured, and the results are presented as the mean±SEM.

FIG. 9 shows the effects of anti-CD11b-I-domain antibody and anti-PD1 antibody combination therapy on the growth of CT26 tumor. Balb/c mice were injected subcutaneously with 3×10⁵ CT26 cells on day 0. When tumor volumes were approximately 50-100 mm³, mice (5 per group) were injected ip with either the control IgG (5 mg/kg), anti-CD11b -I-domain antibody (5 mg/kg), anti-PD1 antibody (5 mg/kg), or anti-CD11b-I-domain antibody (5 mg/kg)+anti-PD1 antibody (5 mg/kg). Injections were repeated every three to four days. Tumor volumes were measured, and the results are presented as the mean±SEM.

FIG. 10 shows the effects of anti-CD11b-I-domain antibody and anti-OX40 antibody combination therapy on the growth of CT26 tumor. Balb/c mice were injected subcutaneously with 3×10⁵ CT26 cells on day 0. When tumor volumes were approximately 50-100 mm³, mice (5 per group) were injected ip with either the control IgG (5 mg/kg), anti-CD11b -I-domain antibody (5 mg/kg), anti-OX40 antibody (5 mg/kg), or anti-CD11b-I-domain antibody (5 mg/kg)+anti-OX40 antibody (5 mg/kg). Injections were repeated every three to four days. Tumor volumes were measured, and the results are presented as the mean±SEM.

FIG. 11 shows the effect of anti-CD11b-I-domain antibody and anti-CD40 antibody combination therapy on the growth of CT26 tumor. Balb/c mice were injected subcutaneously with 3×10⁵ CT26 cells on day 0. When tumor volumes were approximately 50-100 mm³, mice (5 per group) were injected ip with either control IgG (5 mg/kg), anti-CD11b-I-domain antibody (5 mg/kg), anti-CD40 antibody (5 mg/kg), or anti-CD11b-I-domain antibody (5 mg/kg)+anti-CD40 antibody (5 mg/kg). Injections were repeated every three to four days. Tumor volumes were measured, and the results are presented as the mean±SEM.

FIGS. 12A-12C show effects of anti-CD11b-I-domain antibody on dendritic cells in CT26 tumor-bearing mice, as analyzed with FACS. FIG. 12A: classic dendritic cells (DC), FIG. 12B: natural killer dendritic cells (NKDC), and FIG. 12C: plasmacytoid dendritic cells (pDC). Balb/c mice were injected subcutaneously with 3×10⁵ CT26 cells on day 0. When tumor volumes were approximately 50-100 mm³, mice were injected ip with either control IgG (5 mg/kg), or anti-CD11b-I-domain antibody (5 mg/kg). Injections were repeated every three to four days. After the fourth treatment, mice were sacrificed, and tumor associated macrophages were isolated. Amounts of classic dendritic cells, natural killer dendritic cells, and plasmacytoid dendritic cells in the tumor were counted by flow cytometry.

FIG. 13 shows FACS analysis of tumor 4−1BB+PD−1+ neoantigen specific CD8 T cells numbers from CT26 tumor bearing mice. Balb/c mice were injected subcutaneously with 3×10⁵ CT26 cells on day 0. When tumor volumes were approximately 50-100 mm³, mice (5 per group) were injected ip with either control IgG (5 mg/kg), anti-CD11b-I-domain antibody (5 mg/kg), anti-CTLA4 antibody (5 mg/kg), or anti-CD11b-I-domain antibody (5 mg/kg)+anti-CTLA4 antibody (5 mg/kg). Injections were repeated every three to four days. After the fourth treatment, mice were sacrificed, and tumor associated macrophages were isolated. Amounts of 4−1BB+PD−1+ neoantigen specific CD8 T cells in the tumor were counted by flow cytometry.

FIG. 14 shows 77 days after initial tumor inoculation, the surviving mice treated with anti-CD11b-I-domain antibody and anti-CTLA4 antibody (referred to as immunized mice) were injected for a second time with 3×10⁵ parental CT26 cells. Two nonimmunized (naïve) mice were injected in the same manner as a control group. Tumor volumes are the mean±SEM.

FIG. 15 shows the Effect of anti-CD11b antibody and Taxol combination therapy on growth of B16F10 tumor. C57BL/6 mice were injected subcutaneously with 2×10⁵ B16F10 cells on day 0. On day 7, mice were injected ip with either Ctrl IgG (5 mg/kg), anti-mouse CD11b-I-domain antibody (5 mg/kg), Taxol (10 mg/kg)+Ctrl IgG (5 mg/kg), or Taxol (10 mg/kg)+anti-CD11b-I-domain antibody (5 mg/kg). Injections were repeated every three to four day. Tumor volumes were measured, and the results are presented as the mean±SEM.

### DEFINITIONS

The term “CD11b” refers to integrin alpha M (ITGAM), which is a subunit of the heterodimeric integrin αMβ2. The other subunit of integrin αMβ2 is the common integrin β2 subunit known as CD18. Integrin αMβ2 is also called macrophage-1 antigen (Mac-1) or complement receptor 3 (CR3), expressed on the surface of leukocytes, including monocytes, granulocytes, macrophages, dendritic cells, B cells, T cells, and nature killer cells.

“CD11b-I-domain” is also referred to as “CD11b-A-domain” (a Von Willebrand factor (vWF) A-type domain), which is inserted in the β-propeller domain and comprises the following amino-acid sequence (SEQ ID NO:1):

(SEQ ID NO: 1)
DIAFLIDGSGSIIPHDFRRMKEFVSTVMEQLKKSKTLFSLMQYSEEFRIH
FTFKEFQNNPNPRSLVKPITQLLGRTHTATGIRKVVRELFNITNGARKNA
FKILVVITDGEKFGDPLGYEDVIPEADREGVIRYVIGVGDAFRSEKSRQE
LNTIASKPPRDHVFQVNNFEALKTIQNQL.

The term “immune response modulator” refers to an agent that can modulate immune response in a host. The term “immune checkpoint blockade drug” refers to an “immune checkpoint inhibitor” that can relieve immunosuppression via immune checkpoints.

### DETAILED DESCRIPTION

Embodiments of the invention relate to methods for modulating immune responses. Embodiments of the invention are based on reagents binding to the I-domain of CD11b on the tumor-associated myeloid cells (TAMCs) in the tumor microenvironment. In accordance with embodiments of the invention, reagents that bind specifically to the I-domain of CD11b may be antibodies, including monoclonal antibodies, or binding fragments thereof.

In accordance with embodiments of the invention, binding to the I-domain of CD11b with a specific reagent (e.g., an anti-CD11b-I-domain antibody) can induce or trigger immunostimulatory responses. While the I-domain of CD11b is known for its involvement in adhesions, inventors of the present invention have unexpected found that specific bindings of such reagents to the I-domain of CD11b may have one or more of the following effects in the tumor microenvironment: increasing the inflammatory cytokine in the tumor microenvironment, decreasing the population of IDO+ myeloid suppresser cells, up-regulating M1 marker over M2 marker on the tumor associated macrophages, increasing M1:M2 tumor-associated macrophage ratios, promoting differentiation of dendritic cells (DC) (including classic dendritic cells, nature killer dendritic cells (NKDC), and plasmacytoid dendritic cells (pDC)), increasing population of 4−1BB+PD−1+ neoantigen specific CD8 T cells. These effects suggest that specific binding of reagents (e.g., anti-CD11b-I-domain antibodies) to the I-domain of CD11b can induce conversion of cold (non-inflamed) tumor to hot (inflamed) tumor, which may allow enhanced efficacy of immune checkpoint therapy.

Embodiments of the invention will be illustrated with the following specific examples. However, one skilled in the art would appreciate that these specific examples are for illustration only and that other modifications and variations are possible without departing from the scope of the invention.

### Anti-CD11b-I-Domain Antibody Treatment Enhanced Inflammatory Cytokine Release in the Tumor Microenvironment

Prior research had established that CD11b activation negatively regulates TLR-triggered inflammatory responses. Because CD11b is expressed on tumor-associated myeloid cells (TAMCs), we reasoned that blocking CD11b with CD11b-I-domain functions using antibodies may increase inflammatory cytokine releases in the tumor microenvironment. We thus assessed the secretion of proinflammatory cytokine (e.g., TNF-α, IL-6, IL-12, IFN-γ, MCP-1, etc.) in B16F10 tumor after treatments with an anti-CD11b-I-domain antibody.

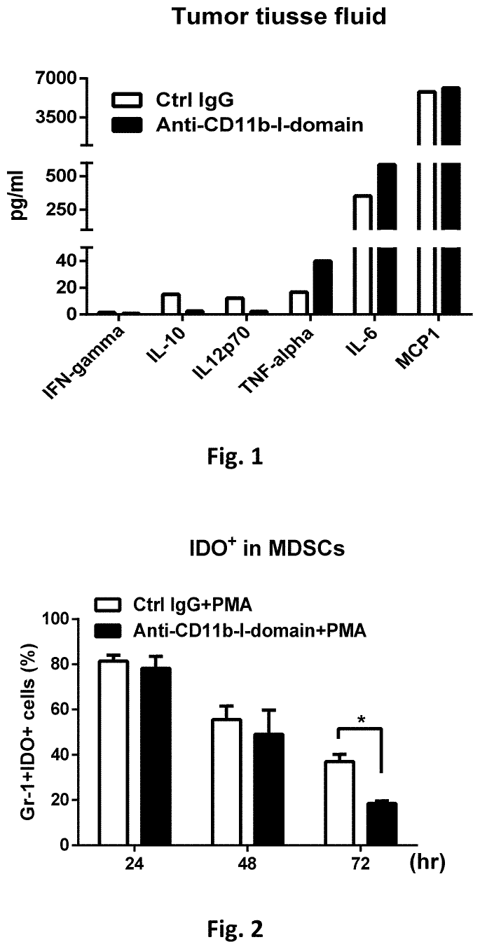

As shown in FIG. 1, the secretions of TNF-α, IL-6, and MCP-1 (monocyte chernoattractant protein 1) are higher in the tissue fluids from anti-CD11b-I-domain antibody-treated tumor, whereas the secretions of IL-10 and IL-12p70 are lower. These results indicate that anti-CD11b-I-domain antibody treatment can increase the production of proinflammatory cytokines. In other words, anti-CD11b-I-domain antibody treatment can convert a cold (non-inflamed) tumor into a hot (inflamed) tumor.

“Hot tumors” are those invaded by T cells, resulting in an inflamed microenvironment. T cells in the tumor microenvironment can be readily mobilized to fight the tumor cells. For example, immune checkpoint blockade drugs (i.e., immune checkpoint inhibitors), such as anti-PD1, anti-PDL1, and anti-CTLA4 antibodies, ran release the brakes exerted by the tumor on the T cells. These drugs work best in “hot” tumors (i.e., those that are inflamed, with high mutagenic burden, and capable of attracting neoantigen specific T-cell infiltration). Therefore, by converting “cold” tumors into “hot” tumors, methods of the invention may enhance the efficacies of immune checkpoint blockade therapies.

### Anti-CD11b-I-Domain Antibody Treatment Reduced the IDO+ Population in Mouse MDSCs and Reversed MDSCs-Induced T Cell Inhibition

Myeloid-derived suppressor cells (MDSCs) are a heterogenous group of immune cells from the myeloid lineage. MDSCs are distinguished from other myeloid cell types in that MDSCs possess strong immunosuppressive activities instead of immunostimulatory properties found in other myeloid cells. Although their mechanisms of action are not fully understood, clinical and experimental evidence indicates that cancer tissues with high infiltration of MDSCs are associated with poor patient prognosis and resistance to therapies.

MDSCs through some mechanisms, such as production of arginase I (arg1) and expression of indoleamine 2,3-dioxygenase (IDO), can induce immunosuppression, leading to T-cell inhibition. In mouse tumor models, MDSCs are found as myeloid cells expressing high levels of CD11b (a classical myeloid lineage marker). Therefore, we set out to investigate the roles of CD11b on MDSCs by studying the effects of CD11b blockade on the MDSC immunosuppression functions. Briefly, MDSCs are isolated from LLC1-bearing mice and treated with anti-CD11b-I-domain antibody. The effects of such treatment on MDSCs properties are assessed.

As shown in FIG. 2, anti-CD11b-I-domain antibody treatment resulted in a significant reduction in the population of IDO+ MDSCs, after stimulation with phorbol 12-myristate-13-acetate (PMA), in a time-dependent manner, as compared with similar treatments with a control IgG. Based on the reduction in IDO+ MDSCs, one would expect that immunosuppression and T-cell inhibition that are mediated by MDSCs should be reduced.

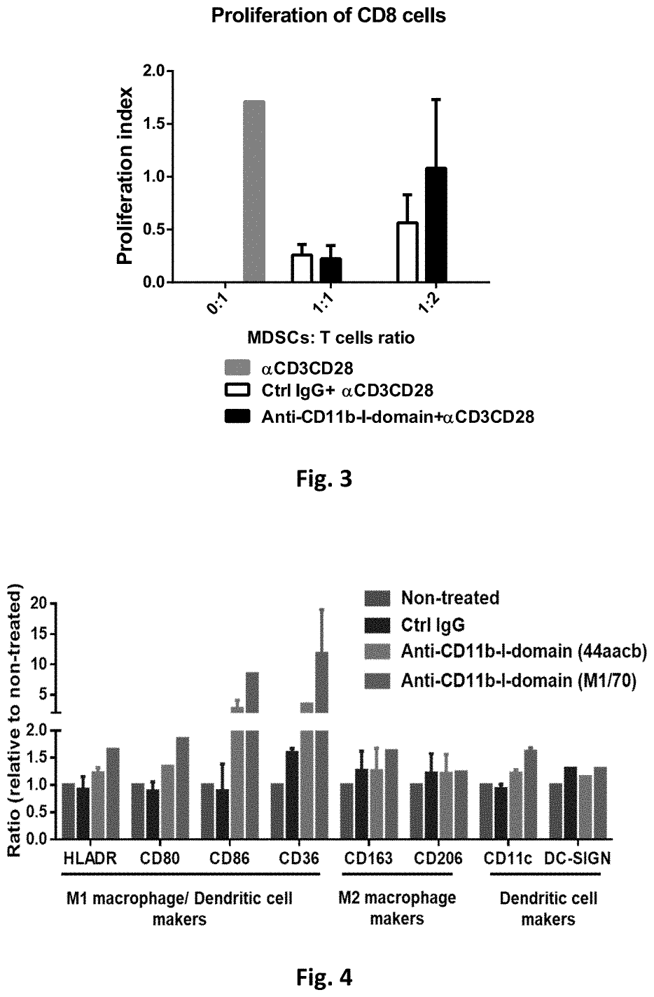

Indeed, as shown in FIG. 3, CD8 cell proliferation in the presence of MDSCs is increased by treatment with an anti-CD11b-I-domain antibody, as compared with the treatment with a control IgG. These results indicate that MDSCs-induced T cell inhibition was significantly reversed when CD11b of MDSCs was blocked by an anti-CD11b-I-domain antibody.

### Anti-CD11b-I-Domain Antibody Treatment Up-Regulated M1 Makers Over M2 Makers

Macrophages are tissue-resident professional phagocytes and antigen presenting cells. Macrophages originate from blood monocytes. In different tissue environments, macrophages undergo specific differentiation into distinct functional phenotypes. They have been commonly divided into two classes: classically activated (M1) macrophages and alternatively activated (M2) macrophages. M1 macrophages encourage inflammation, whereas M2 macrophages decrease inflammation and encourage tissue repair. This difference is reflected in their metabolisms: M1 macrophages can metabolize arginine to generate nitric oxide, whereas M2 macrophages metabolize arginine to produce ornithine.

Phenotypically, M1 macrophages express high levels of major histocompatibility complex class II (MHC II), CD36, and co-stimulatory molecules CD80 and CD86. In contrast, M2 macrophages have been characterized as CD163+ and CD206+. Tumor associated macrophages (TAMs) display an M2-like phenotype and promote tumor progression. To examine whether anti-CD11b-I-domain antibody treatment can skew tumor-associated macrophages towards the M1 phenotype, human macrophages were differentiated from PBMCs in vitro in the presence of A549 lung cancer cells.

As shown in FIG. 4, the expressions of M1 markers are substantially higher in the anti-CD11b-I-domain antibody treatment groups (anti-CD11b (44aacb) and anti-CD11b (M1/70)), as compared with the control IgG treatment group. On the other hand, the expressions of M2 markers showed no or only slight enhancement in the anti-CD11b-I-domain antibody treatment groups, as compared with the control IgG treatment group. In addition, anti-CD11b-I-domain antibody treatment also up-regulated CD11c and DC-SIGN, which are dendritic cell markers. Together, these results demonstrated that CD11b blockade skew the tumor associated macrophage towards the M1-phenotype and mature dendritic cells, leading to an inflammatory microenvironment conducive to immunotherapy.

This experiment used two different anti-CD11b-I-domain antibodies (i.e., 44aacb and M1/70), which are commercially available. Anti-CD11b antibody 44aacb is available from many commercial sources, such as Novus Biologicals (Littleton, Colo., USA) and ATCC. Anti-CD11b antibody M1/70 is available from Thermo Fisher, Abcam, BioLegent, etc. Furthermore, other anti-CD11b antibodies can also be used. The results from these experiments indicate that the effects are not restricted to any particular antibody. In fact, any antibody, or a binding fragment thereof, that can bind to CD11b I-domain can be used with embodiments of the invention.

### Anti-CD11b-I-Domain Antibody Treatment Switches the Activation of Tumor Associated Macrophages from an Immunosuppressive M2-Like to a More Inflammatory M1-Like State

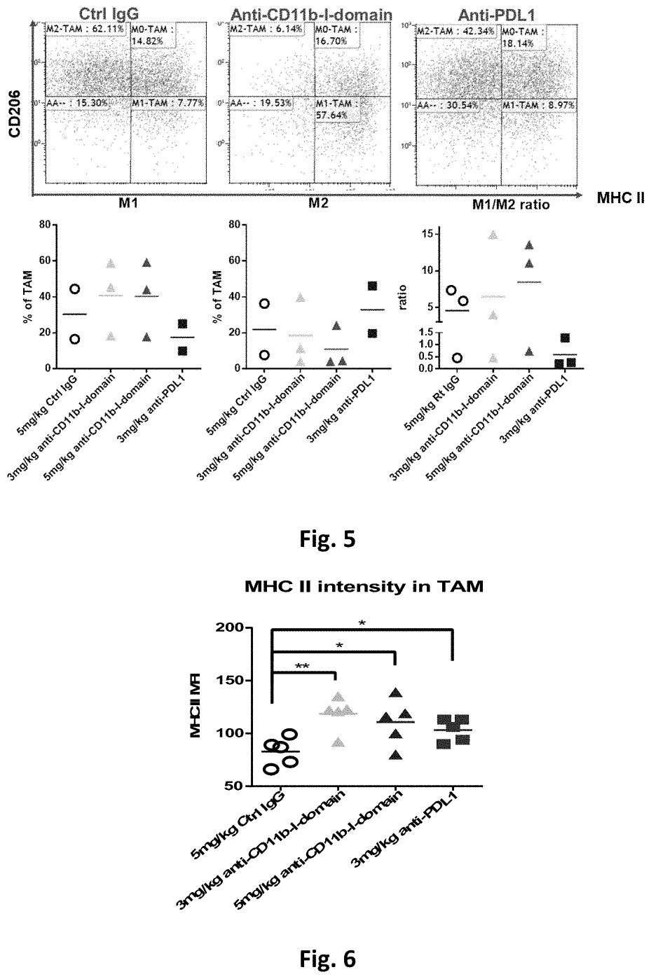

As discussed above, CD11b blockade skews macrophages towards the M1 phenotype in vitro. We further confirmed this observation in a CT26 tumor model. Analysis of tumor infiltrated leukocytes in the CT26 tumor bearing mice shows that treatment with anti-CD11b-I-domain antibody increased the M1/M2 macrophage ratio and increased mature dendritic cell population (FIG. 5) and markedly increased the expression of MHC II (FIG. 6) in the tumor associated macrophages, as compared with treatments with a control IgG. These results suggest an enhanced antigen presentation capacity. Taken together, these results show that modulating the suppressive phenotype of tumor associated macrophages towards a more immune active one can be achieved by CD11b-I-domain blockade.

### Synergistic Effect of Anti-CD11b-I-Domain Antibody and TLR Agonist Treatment in Antitumor Immunity

Results from recent research show that high avidity ligation of CD11b-I-domain leads to rapid inhibition of Toll-like receptor (TLR) signaling. Thus, blocking the CD11b-I-domain activity with anti-CD11b-I-domain antibody may reverse the inhibition of TLR signaling. We next examine whether combination immunotherapy with CpG oligonucleotide (TLR9 agonist) and CD11b blockade can enhance the antitumor efficacy. Balb/c female mice were implanted subcutaneously with 3×10⁵ CT26 colon cancer cells. When tumor volumes were approximately 50-100 mm³, mice were injected ip with a control IgG, an anti-CD11b-I-domain antibody at 5 mg/kg, a CpG oligonucleotide at 50 μg, or a combination of 5 mg/kg of anti-CD11b-I-domain antibody and 50 μg of CpG oligonucleotide.

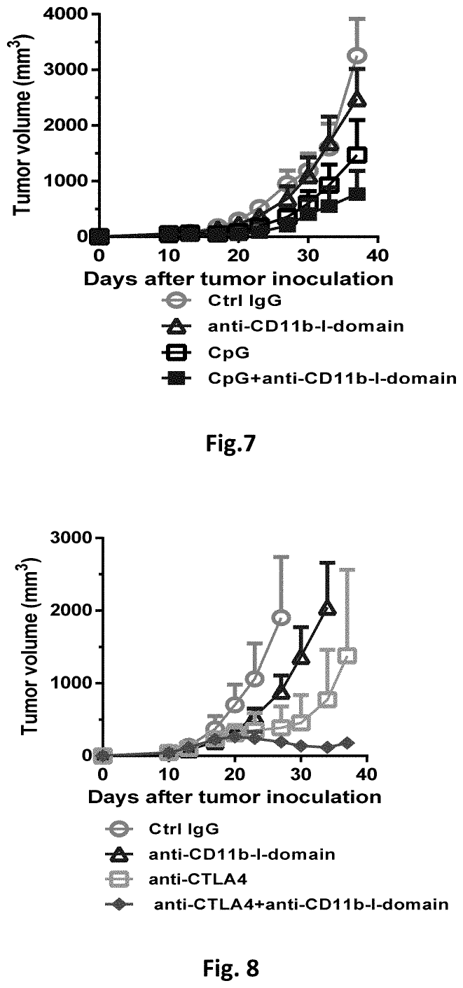

As shown in FIG. 7, monotherapy with CpG oligonucleotide inhibited tumor growth. Significantly, mice treated with the combination of anti-CD11b-I-domain antibody and CpG oligonucleotide had the best antitumor response. The dramatic effects of the combination therapy suggest the existence of a synergistic effect.

While the above experiment uses CpG oligonucleotide (TLR9 agonist) as an example, other TLR agonists may also be used in a similar manner. One skilled in the art would appreciate that the anti-CD11b reagents of the invention may also be used with these other TLR agonist approaches.

### Synergistic Effect of Anti-CD11b-I-domain Antibody and Immune Checkpoint Treatment in Antitumor Immunity

As noted above, by binding specifically to the I-domain of CD11b, methods of the invention may convert “cold” tumors into “hot” tumors, thereby enhancing the efficacy of immune checkpoint blockade therapy. We next investigate the effects of such combination therapy.

CTLA4 is an inhibitory receptor expressed by T-cells and negatively regulates the effector phase of T-cell response after ligation (ligand binding) of CD80/CD86 expressed on the dendritic cells or macrophages. Because anti-CD11b-I-domain antibody treatment enhances the expression of CD80/CD86 on the tumor associated macrophages, we next examine whether combination immunotherapy with CD11b and CTLA4 blockade can enhance the antitumor efficacy. Balb/c female mice were implanted subcutaneously with 3×10⁵ CT26 colon cancer cells. When tumor volumes were approximately 50-100 mm³, mice were injected ip with a control IgG, an anti-CD11b-I-domain antibody at 5 mg/kg, an anti-CTLA4 antibody at 5 mg/kg, or a combination of 5 mg/kg of anti-CD11b-I-domain antibody and 5 mg/kg of anti-CTLA4 antibody.

As shown in FIG. 8, monotherapy with anti-CD11b-I-domain antibody was partially efficacious, while monotherapy with anti-CTLA4 antibody significantly inhibited tumor growth. Significantly, mice treated with the combination of anti-CD11b-I-domain antibody and anti-CTLA4 antibody had the best antitumor response, resulting in a 60% regression rate. The dramatic effects of the combination therapy suggest the existence of a synergistic effect.

While the above experiment uses CTLA4 as an example, other immune checkpoint targets may also be used in a similar manner. For example, PD-1 and PD-L1 have been shown to be involved in immune checkpoint regulations and antibodies against PD-1 and PD-L1 have been shown to be effective in reversing immune suppression. OX40 (also known as CD134 or tumor necrosis factor receptor superfamily member 4 (TNFFRSF4)) and T-cell immunoglobulin and mucin-domain containing-3 (TIM3) are other examples of immune checkpoints. Blockage of OX40 or TIM3 can relieve tumor-induced immune suppression.

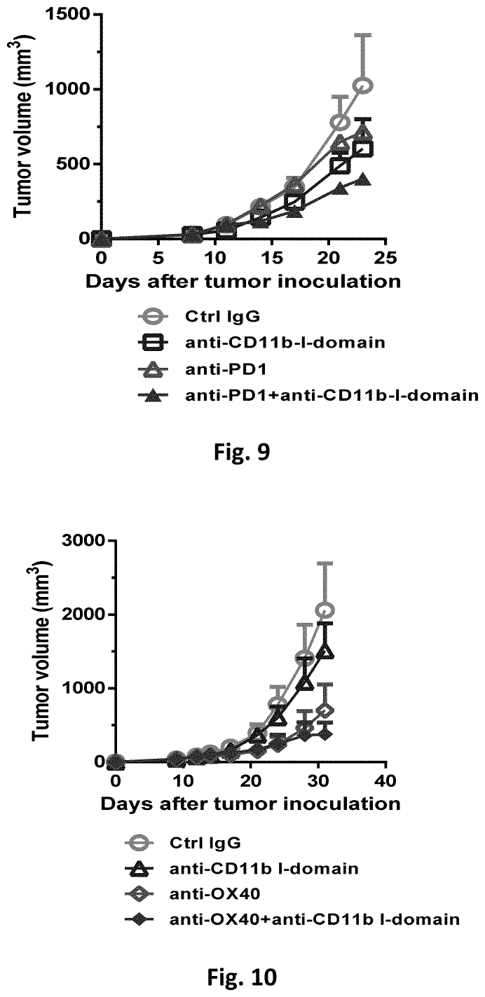

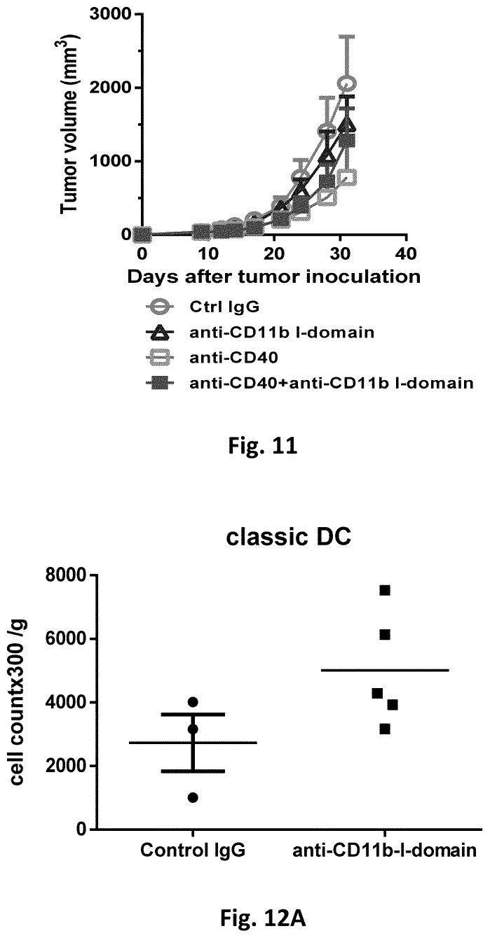

As shown in FIG. 9, monotherapy with anti-PD1 antibody slightly inhibited tumor growth, while mice treated with the combination of anti-CD11b-I-domain antibody and anti-PD1 antibody had the best antitumor response. Similarly, anti-OX40 or anti-CD40 antibody combined with anti-CD11b-I-domain antibody had the best antitumor response (FIG. 10 and FIG. 11). One skilled in the art would appreciate that the anti-CD11b reagents of the invention may also be used with these other immune checkpoint blockage approaches.

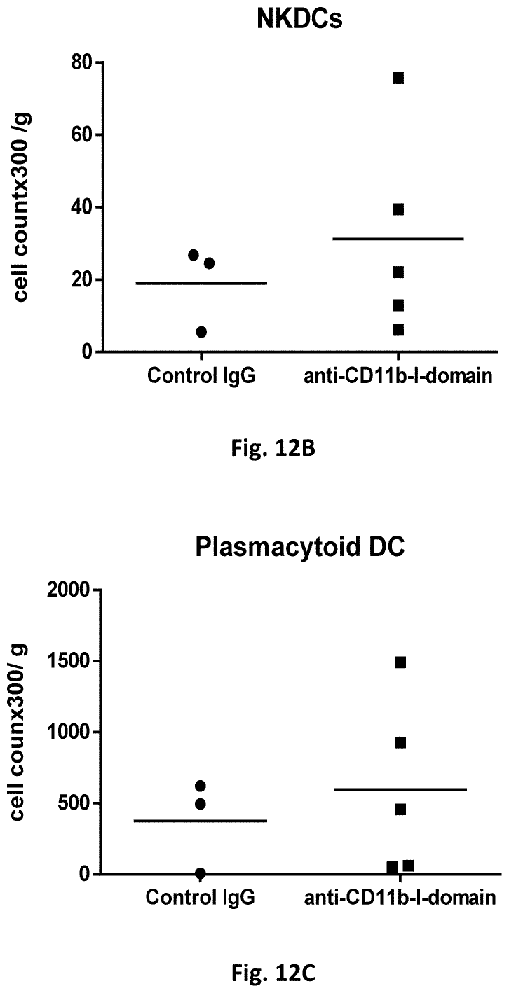

Dendritic cells (DCs) are efficient antigen-presenting cells and are promising option for improvement of therapeutic vaccines. As shown in FIGS. 12A-12C, treatment with anti-CD11b-I-domain antibody increased the numbers of classic dendritic cells (DC) (FIG. 12A), natural killer dendritic cells (NKDC) (FIG. 12B), and plasmacytoid dendritic cells (pDC) (FIG. 12C) in the tumor microenvironment.

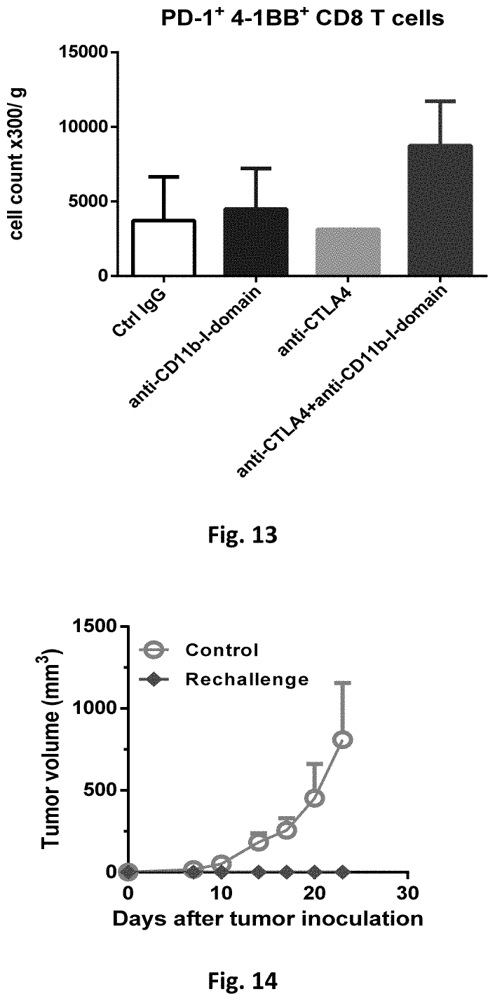

In addition, as shown in FIG. 13, treatment with anti-CD11b-I-domain antibody alone modestly increased the number of effector PD−1⁺4−1BB⁺ neoantigen specific CD8 T cells in the tumor microenvironment, while treatment with anti-CTLA4 antibody alone had little effect. In contrast, the combination treatment with anti-CD11b-I-domain antibody and anti-CTLA4 antibody markedly increased the number of effector PD−1⁺4−1BB⁺ neoantigen specific CD8 T cells in the tumor microenvironment, exhibiting a remarkable synergistic effect (FIG. 13). Taken together, these results show that modulating tumor microenvironment, i.e., converting the immunosuppressive tumor microenvironment towards a more immunostimulatory one, can be achieved by CD11b-I-domain blockade (e.g., binding of an antibody to CD11b-I-domain). As a result, anti-CD11b-I-domain antibody can enhance the efficacies of immunotherapy agents, such as immune checkpoint blockage drugs: anti-PD1, anti-PDL1, and/or anti-CTLA4 antibodies.

### Long-Term Memory Effects of CD11b-I-domain Blockade

Immune checkpoint blockade drugs, such as anti-PD1, anti-PDL1, and anti-CTLA4 antibodies, can elicit durable clinical responses in cancer patients. Therefore, we also investigate the long-term effects of anti-CD11b-I-domain treatment.

Briefly, 77 days after the initial tumor inoculation and treatment with a combination of anti-CD11b-I-domain antibody and anti-CTLA4 antibody (referred to as immunized mice), the surviving mice were injected for a second time with 3×10⁵ parental CT26 cells (colon cancer cells). Two naïve (not previously immunized and treated) mice were injected in the same manner as a control group. The mice were monitored, and tumor volumes were measured following the inoculation.

As shown in FIG. 14, tumor grew rapidly in the control group (naïve mice). In contrast, the previously immunized and treated survivors retained the ability to confine tumor growth, indicating that blockade of CD11b I-domain (e.g., with an anti-CD11b-I-domain antibody) can elicit long-term responses.

### Synergistic Effect of Anti-CD11b-I-Domain Antibody and Chemotherapy Treatment in Antitumor Immunity

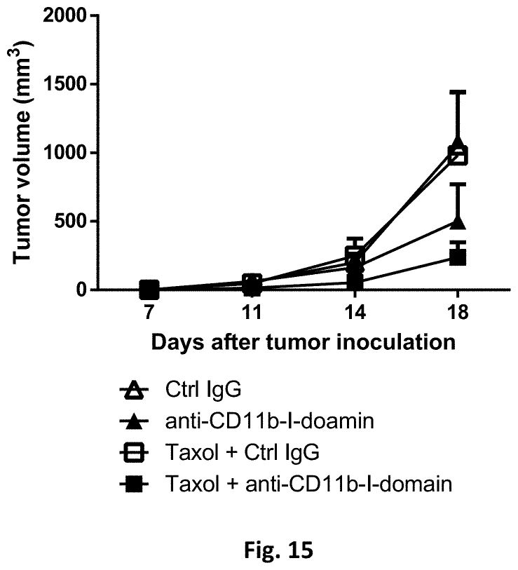

We next examine whether combination immunotherapy with chemotherapy and CD11b-I-domain blockade can enhance the antitumor efficacy. C57BL/6 female mice were implanted subcutaneously with 2×10⁵ B16F10 melanoma cancer cells on day 0. On day 7, mice were injected ip with a Ctrl IgG at 5 mg/kg, an anti-CD11b-I-domain antibody at 5 mg/kg, a combination of 5 mg/kg of Ctrl IgG and 10 mg/kg of Taxol, or a combination of 5 mg/kg of anti-CD11b-I-domain antibody and 10 mg/kg of Taxol. Injections were repeated every three to four day. Significantly, mice treated with the combination of anti-CD11b-I-domain antibody and taxol had the best antitumor response (FIG. 15). The dramatic effects of the combination therapy suggest the existence of a synergistic effect.

Taxol (paclitaxel) functions as a chemotherapeutic agent mainly through its ability to bind the microtubule to act as a mitotic inhibitor. However, Taxol has also been found to have activity in activating lymphocytes, including T cells, B cells, NK cells, and dendritic cells. Thus, Taxol may also be considered as an immune response modulator.

Radiotherapy may potentiate the efficacy of immune response modulator via several mechanisms includes inducing tumor cell apoptosis, thereby increasing tumor antigens presentation via APCs and direct T cell activation. Radiotherapy induced tumoricidal effect results in release of more tumor antigens leading to clonal expansion of activated T cells through which both the diversity of T cell populations and the rate at which they are activated are enhanced

Oncolytic viruses can directly lyse tumor cells, leading to the release of soluble antigens, danger signals and type I interferons, which drive antitumor immunity. In addition, some oncolytic viruses can be engineered to express therapeutic genes or can functionally alter tumor-associated endothelial cells, further enhancing T cell recruitment into immune-excluded or immune-deserted tumor microenvironments.

While the above experiment uses Taxol as an example, other chemotherapy reagents may also be used in a similar manner. One skilled in the art would appreciate that the anti-CD11b reagents of the invention may also be used with these other chemotherapy approaches.

The above experiments clearly show that blocking the I-domain of CD11b can convert the tumor microenvironment into more inflammatory state that is more conducive to immune therapy approaches, as evidenced by: increased inflammatory cytokine in the tumor microenvironment, decreased population of IDO+ myeloid suppresser cells, up-regulated M1 marker over M2 marker on the tumor associated macrophage, increased M1:M2 tumor associated macrophage ratio, enhanced differentiation of dendritic cells (DC), nature killer dendritic cells (NKDC), and plasmacytoid dendritic cells (pDC), increased population of 4−1BB+PD−1+ neoantigen specific CD8 T cells. These properties can be used to enhance the immunotherapeutic efficacy. Indeed, combination therapies using anti-CD11b antibodies and another antibody targeting an immune checkpoint can achieve dramatic synergistic effects. These combination therapies will be most beneficial for cancer therapy. CD11b I-domain is known to be involved in adhesion functions. The finding that blockage of the I-domain of CD11b can convert the tumor microenvironment into a more inflammatory state conducive for induction of immune responses is truly unexpected.

Embodiments of the invention may be practiced with any suitable methods/procedures known in the art. The following will illustrate specific examples for embodiments of the invention. However, one skilled in the art would appreciate that these specific examples are for illustration only and that other modifications and variations are possible without departing from the scope of the invention.

### Human Cell Isolation and Cell Line

Human PBMC were isolated from healthy volunteer donors by venipuncture. Written informed consent was obtained for participation in the study, which was approved by the Institutional Review Board of the Mackay Memorial Hospital. Human monocytes were isolated using methods known in the art. Briefly, peripheral blood mononuclear cells (PBMCs) were isolated using Ficoll-Paque Plus (GE Healthcare) gradient centrifugation.

A549 lung cancer cell line was obtained from the American Type Culture Collection (ATCC) and cultured in F-12K medium with 10% fetal calf serum (Hyclone, Inc., Logan, Utah). All cell lines were maintained at 37° C. in complete medium (RPMI-1640 with 10% fetal calf serum, 2 mM L-Glutamine, 100 U/mL Penicillin, and 100 μg/mL Streptomycin). Cells were grown in tissue culture flasks in humidified, 5% CO₂ incubators, and passaged 2-3 times per week by light trypsinization.

### Animal and Tumor Cell Line

Balb/c mice (6 to 8 weeks old) were purchased from the National Laboratory Animal Center (Taipei, Taiwan). All animal experiments were performed under specific pathogen-free conditions and in accordance with guidelines approved by the Animal Care and Usage Committee of Mackay memorial hospital (Taipei, Taiwan). The body weight of each mouse was measured at the beginning of treatment and every day during the treatment period. CT26 cells are murine colon cancer cells derived from Balb/c mice. B16F10 cells are murine melanoma cancer cells derived from C57/BL6 mice. Cells were maintained in Dulbecco's modified Eagle's medium (DMEM), 10% heat-inactivated fetal calf serum, 2 mM L-glutamine, penicillin (100 U/ml), and streptomycin (100 μg/ml) at 37° C. in a 5% CO₂ humidified atmosphere.

### Antibodies and Reagents

### For Human PBMC Study

The hybridoma of the monoclonal anti-CD11b-I-domain Antibody (44aacb) was purchased from ATCC. Antibody produced from this hybridoma was purified using protein A-conjugated sepharose. Mouse IgG2a used as a control antibody was purchased from Biolegend (San Diego, Calif.).

### For Murine Cancer Model

Rat antibody specific to mouse/human CD11b-I-domain (clone M1/70), rat antibody specific to murine PD1 (clone RMP1-14), rat antibody specific to murine OX40 (clone OX-86), rat antibody specific to murine CD40 (clone FGK4.5), rat control IgG2b antibody (clone LTF-2), Syrian hamster anti-murine CTLA4 (clone 9H10), and Syrian hamster control IgG were purchased from BioXcell (West Lebanon, N.H.). CpG oligonucleotide (class B, ODN 1668) was purchased from Invivogen (San Diego, Calif.). Taxol was obtained from MacKay Memorial Hospital.

### Tumor-Associated Myeloid Suppressor Cells Generation Protocol

### i. Induction

Human PBMC were isolated from healthy volunteer donors by venipuncture (60 mL total volume), followed by differential density gradient centrifugation (Ficoll Hypaque, Sigma, St. Louis, Mo.). PBMC were cultured in complete medium (1×10⁶ cells/mL) in 24-well plates with human tumor cell lines at a 40:1 ratio for five to six days. For antibody-treatment experiments, PBMC-tumor cell line co-cultures were repeated in the presence or absence of the antibodies, including anti-mouse/human CD11b-I-domain (clone M1/70, BioXcell), anti-human CD11b-I-domain (clone 44aacb, hybridoma from ATCC), mouse IgG2a isotype control (clone MG2a-53, Biolegend), and rat IgG2b isotype control (clone LTF-2, BioXcell).

### ii. Myeloid Suppressor Cells Isolation

After 5 days, all cells were collected from tumor-PBMC co-cultures. Adherent cells were removed using the non-protease cell detachment solution Detachin™ (GenLantis, San Diego, Calif.). Myeloid cells were then isolated from the co-cultures using anti-CD33 magnetic microbeads and LS column separation (Miltenyi Biotec, Germany) as per manufacturer's instructions. Purity of the isolated cell populations was found to be greater than 90% by flow cytometry and viability of the isolated cells was confirmed using trypan blue dye exclusion.

### iii. Suppression Assay

The suppressive function of tumor-educated myeloid cells was measured by their abilities to inhibit proliferation of allogeneic T cells in a Suppression Assay as follows: T cells isolated from healthy donors by Pan T isolation kit (Miltenyi Biotec, Auburn, Calif.) were Carboxyfluorescein succinimidyl ester (CFSE)-labeled (2.5 μM, Invitrogen) and seeded in 96-well plates with previously isolated myeloid cells at 1×10⁵ cells/well at the 1:1 ratio. T cell proliferation was induced by anti-CD3/CD28 stimulation beads (ThermoFisher scientific, Carlsbad, Calif.) or coated anti-CD3 (clone OKT3) antibodies. Suppression Assay wells were analyzed with flow cytometry for T cell proliferation after three days. Controls included a positive T cell proliferation control (T cells alone with CD3/CD28 stimulation) and an induction negative control (medium only). Samples were run on a FACSCalibur flow cytometer (BD Biosciences, San Jose, Calif.), and data acquisition and analysis were performed using CellQuestPro software (BD).

### Characterization of Human Myeloid Suppressor Cells

### i. Flow Cytometry Analyses of Cell Phenotypes

The phenotype of in vitro-generated myeloid suppressor cells was examined for expression of myeloid, antigen-presenting, and suppressor cell markers. For staining, cells were collected from 24 well-plate using Detachin™ to minimize cell surface protein digestion, and washed twice with FACS buffer (2% FCS in PBS) before resuspending 10⁶ cells in 100 μl FACS buffer. Cells were treated with Fc blocker (Human BD Fc Block) and stained for 20 mins with cocktails of fluorescently-conjugated monoclonal antibodies or isotype-matched controls. For intracellular staining, cells were fixed and permeabilized using Fixation/Permeabilization Kit (BD) after surface staining. Antibodies used were purchased either from BD Biosciences: CD11c (clone Bu15), CD33 (clone HIM3-4), HLA-DR (clone L243), CD11b (clone ICRF44), CD86 (clone 2331), CD80 (clone L307.4), CD56 (clone B159), CD206 (clone 19.2), DC-SIGN (clone DCN46), 7-AAD; or Biolegned: HLA-DR (clone L243), CD163 (clone RM3/1), CD68 (clone Y1/82A); or R&D systems: IDO (clone 700838). These antibodies are examples, and any other suitable antibodies may be used. For example, any anti-CD11b antibodies that bind the I-domain may be used (e.g., Anti-CD11b (44aacb clone), anti-CD11b (M1/70 clone, etc.). Such anti-CD11b antibodies may include those newly generated or those obtained from commercial sources (e.g., BD Biosciences, Abcam, Thermo Fisher Scientific, etc.).

Samples were run on a BD FACSCalibur flow cytometer and data acquisition and analysis were performed as described above. Data are from three to six unique donors. PBMC cultured in medium alone were run in parallel for comparison.

### ii. Measurement of Cytokine/Chemokine by Cytometric Bead Array

Tumor tissue fluids were collected from B16F10 tumor after anti-CD11b-I-domain antibody treatment and stored in aliquots at −20° C. Levels of IFN-gamma, MCP-1, IL-6, TNFα, IL12p70, and IL-10 in samples were measured using mouse inflammatory cytokine cytometric bead array kit (BD) per manufacturer's instructions.

### Protocol of Cancer Treatment

### Subcutaneous Tumor Model

Balb/c mice were inoculated subcutaneously with 3×10⁵ CT26 cells. When tumor volumes were approximately 50-100 mm³, treatment was started. Tumor-bearing mice were treated intraperitoneally (ip) with different antibodies twice per week. Mice were monitored and scored for the formation of palpable tumors twice weekly and sacrificed if tumors exceeded the predetermined size of 3,000 mm³. Tumor volumes were measured with calipers and calculated with the following formula: A×B²×0.54, where A is the largest diameter, and B is the smallest diameter.

### Tumor Dissociation and Cell Population Analysis

Balb/c tumors were harvested, weighted, and finely cut into pieces using surgical scalpels and further enzymatically dissociated using a tumor dissociation kit (Miltenyi Biotec) according to the manufacturers' instructions and using the Gentle MACS dissociator (Miltenyi Biotech). Single-cell suspensions of tumors were resuspended in PBS supplemented with 1% FCS, and erythrocytes were lysed. Non-specific labeling was blocked with anti-CD16/32 (Fc Block; BD) before specific labeling. Cells were stained with the following rat-anti-mouse Abs from BioLegend: anti-CD8a fluorescein isothiocyanate (FITC), anti-CD8b FITC, anti-Gr1 FITC, anti-CD86 FITC, anti-CD206 phycoerythrin (PE), anti-CD80 PE-Dazzle594, anti-CD11b-I-domain PerCP-Cy5.5, anti-PDL1 allophycocyanin (APC), anti-CD45 BV510, anti-F4/80 Alexa 700, anti-IAIE APC-Cy7, anti-Ly6C PECy7, anti-CD11c Alexa 700, anti-Ly6G PE-Dazzle594, anti-IDO AF647, anti-CD335 BV421, and anti-CD3e PE Dazzle. Fixable viability dyes (eBioscience™ Fixable Viability Dye eFluor™ 450) was used for live-dead cell discrimination. The samples were analyzed using a BECKMAN COULTER Gallios flow cytometer and analyzed with Kaluza® software.

### In Vitro Mouse MDSCs Isolation and Suppression Assay

Spleens were collected from LLC1 tumor-bearing mice. Splenocytes were harvested and Myeloid-Derived Suppressor Cells (MDSCs) were isolated using Myeloid-Derived Suppressor Cell Isolation Kit and LS column separation (Miltenyi Biotec) per manufacturer's instructions. Purity of the isolated cell populations was found to be greater than 90% by flow cytometry, and viability of the isolated cells was confirmed using trypan blue dye exclusion. Indoleamine 2,3-dioxygenase (IDO) expression in MDSCs stimulated with phorbol 12-myristate-13-acetate (PMA) for 24 hrs. to 72 hrs. were evaluated by cellular surface staining with anti-mouse Gr-1 FITC antibody and intracellular staining with anti-mouse IDO APC antibody. T cells were collected from splenocytes of naïve mice and isolated using anti-mouse CD90.2 magnetic particles (BD IMag). CF SE-labeled T cells were co-cultured with MDSCs at 1:1 or 1:2 ratio in the absent or present of antibodies, including anti-mouse/human CD11b (clone M1/70, BioXcell) and rat IgG2b isotype control (clone LTF-2, BioXcell). T cell proliferation was induced by anti-CD3/CD28 stimulation antibodies.

### Statistical Analysis

Data were analyzed using Prism 6.0 (GraphPad) and expressed as the mean±SEM. Comparisons between groups were performed using the Student t test. Correlations were determined using the Pearson's correlation coefficient. A p value <0.05 was considered significant.

1. A pharmaceutical composition for use in treating cancer by modulating an immune response, comprising a reagent that binds specifically to the I-domain of CD11b on cells.

2. The pharmaceutical composition according to claim 1, wherein the CD11b is on tumor-associated myeloid cells (TAMCs).

3. The pharmaceutical composition according to claim 1, wherein the reagent is an antibody that binds the I-domain of CD11b.

4. The pharmaceutical composition according to claim 1, wherein the pharmaceutical composition further comprises an immune response modulator.

5. The pharmaceutical composition according to claim 4, wherein the immune response modulator is a reagent that binds specifically to PD-1, PD-L1, CTLA4, CD40, OX40, or a toll-like receptor (TLR).

6. The pharmaceutical composition according to claim 3, wherein the immune response modulator is an anti-PD-1 antibody, an anti-PD-L1 antibody, an anti-CTLA4 antibody, an anti-CD40 antibody, an anti-OX40 antibody, a toll-like receptor agonist, an oncolytic virus, a radiotherapy, or a chemotherapeutic agent.

7. The pharmaceutical composition according to claim 3, wherein the immune response modulator is an anti-CTLA4 antibody.

8. The pharmaceutical composition according to claim 6, wherein the toll-like (TLR) receptor agonist is CpG.

9. The pharmaceutical composition according to claim 6, wherein the chemotherapeutic agent is taxol.

10. A method for modulating an immune response, comprising administering a pharmaceutical composition to a subject in need thereof, wherein the pharmaceutical composition comprises a reagent that binds specifically to the I-domain of CD11b on cells.

11. The method according to claim 10, wherein the CD11b is on tumor-associated myeloid cells (TAMCs).

12. The method according to claim 10, wherein the reagent is an antibody that binds the I-domain of CD11b.

13. The method according to claim 10, wherein the pharmaceutical composition further comprises an immune response modulator.

14. The method according to claim 13, wherein the immune response modulator is a reagent that binds specifically to PD-1, PD-L1, CTLA4, CD40, OX40, or a toll-like receptor (TLR).

15. The method according to claim 12, wherein the immune response modulator is an anti-PD-1 antibody, an anti-PD-L1 antibody, an anti-CTLA4 antibody, an anti-CD40 antibody, an anti-OX40 antibody, a toll-like receptor agonist, an oncolytic virus, a radiotherapy, or a chemotherapeutic agent.

16. The method according to claim 12, wherein the immune response modulator is an anti-CTLA4 antibody.

17. The method according to claim 15, wherein the toll-like (TLR) receptor agonist is CpG.

18. The method according to claim 15, wherein the chemotherapeutic agent is taxol.

19. The method according to claim 11, wherein the reagent is an antibody that binds the I-domain of CD11b.

20. The method according to claim 11, wherein the pharmaceutical composition further comprises an immune response modulator.
